(19) United States
(12) Patent Application Publication
(10) Pub. No.: US 2025/0288712 A1
(43) Pub. Date: Sep. 18, 2025

(54) MULTIFUNCTIONAL CLEANING AND DISINFECTING DEVICE

(71) Applicant: Patrick Orrin Spears, Brentwood, CA (US)

(72) Inventor: Patrick Orrin Spears, Brentwood, CA (US)

(73) Assignee: CleanNesta LLC, Brentwood, CA (US)

(21) Appl. No.: 18/606,450

(22) Filed: Mar. 15, 2024

(51) Int. Cl. A61L 2/24; A61L 2/07; A61L 2/22

(52) U.S. Cl. CPC A61L 2/24; A61L 2/07; A61L 2/22; A61L 2202/11; A61L 2202/14; A61L 2202/17

(57) ABSTRACT

A multifunctional cleaning and disinfecting device comprising a pressure and flow unit and a number of cleaning modules mounted inside a compact and ultraportable housing. A pressure regulator and a flow control valve of the pressure and flow unit independently control pressure and flow rate of a fluid coming from a tank located inside the housing or from an outside supply. A flow diverter of the pressure and flow unit diverts the regulated flow of fluid to a desired cleaning module. A system control module configures a cleaning module selected by a user to perform one or more cleaning and disinfecting functions on a surface as per the user's instructions received through a user interface.

### FIELD OF THE INVENTION

The present invention in general is related to the field of cleaning devices. More particularly, the present invention is related to cleaning and disinfecting systems having multiple, configurable cleaning and disinfecting functions.

### BACKGROUND OF THE INVENTION

Different types of surfaces require different types of cleaning methods and techniques and, generally, separate cleaning devices are available for such specific purposes. The high degree of specificity in each cleaning devices' design and function often makes these devices unsuitable for use across varying surfaces and spaces. In essence, these devices are designed or intended for a specific surface or space. Also, conventional commercial cleaning devices are often large, bulky and difficult to maneuver. Additionally, if more than one type of cleaning device is required at a jobsite, it can further become a more labor, time and cost intensive operation to transport, navigate and/or maintain multiple different cleaning machines.

Thus, there exists a need for a multifunctional cleaning and disinfecting device which can overcome the above-mentioned problems associated with the need for multiple different cleaning devices.

### OBJECT OF THE INVENTION

An object of the present invention is to provide a multifunctional cleaning and disinfecting device.

Yet another object of the present invention is to provide a multifunctional cleaning and disinfecting device which is compact, lightweight and ultraportable.

Still another object of the present invention is to provide a highly efficient and effective multifunctional cleaning and disinfecting device.

Another object of the present invention is to provide a multifunctional cleaning device which can carry out cleaning and disinfecting functions in different configurations, environments and disparate locations.

Yet another object of the present invention is to provide a multifunctional cleaning and disinfecting device having a user friendly interface.

Another object of the present invention is to provide a plug and play configurable multifunctional cleaning and disinfecting device.

Yet another object of the present invention is to provide a compact fluid pressure and flow rate regulator.

These as well as other objects of the present invention are apparent upon inspection of this specification, including the drawings attached hereto.

### SUMMARY OF THE INVENTION

The following presents a simplified summary in order to provide a basic understanding of some aspects of the disclosed invention. This summary is not an extensive overview, and it is not intended to identify key/critical elements or to delineate the scope thereof. Its sole purpose is to present some concepts in a simplified form as a prelude to the more detailed description that is presented later.

Present invention is directed to a multifunctional cleaning and disinfecting device. It comprises a configurable pressure flow unit and a number of cleaning and disinfecting modules installed inside a compact, lightweight and ultraportable housing. A configurable pressure regulator and a flow control valve of the pressure flow unit to independently control pressure and flow rate of a fluid coming optionally from a mounted water supply tank located inside the housing, or from a direct water supply source located outside the housing. A flow diverter of the pressure flow unit that diverts the regulated pressure and flow of fluid to a desired cleaning and disinfecting module. A system control module configures a cleaning and disinfecting module selected by a user to perform one or more cleaning and disinfecting functions on a surface or space as per the user's instructions received through a user interface.

When an instant steam generator cleaning module is present and selected, the system control module configures it to generate an adjustable steam flow from the flow of fluid. Similarly, when a chemical injection cleaning module is present and selected, it takes in a desired chemical from detachable bottles and dynamically and proportionately mixes the chemical with the flow of fluid for application at a desired flow rate and pressure. If the selected cleaning module is a vacuum blower unit then it can be configured to generate an adjustable negative pressure or an adjustable positive pressure, as required. One of the cleaning modules can also be a disinfector configured to deliver an electrically charged aerosol mist using the fluid coming through the pressure flow unit.

A number of applicator parts can be optionally attached or detached to the housing to extend the release of the fluid or air from the different cleaning modules. One or more hand and floor cleaning and disinfecting attachments are attachable as the applicators required.

### BRIEF DESCRIPTION OF DRAWINGS

In order to describe the manner in which features and other aspects of the present disclosure can be obtained, a more particular description of certain subject matter will be rendered by reference to specific embodiments which are illustrated in the appended drawings. Understanding that these drawings depict only typical embodiments and are not therefore to be considered to be limiting in scope, nor drawn to scale for all embodiments, various embodiments will be described and explained with additional specificity and detail through the use of the accompanying drawings in which:

FIG. 1A is a front left perspective view of the multifunctional cleaning and disinfecting device in accordance with an embodiment of the present invention;

FIG. 1B is a front right perspective view of the multifunctional cleaning and disinfecting device in accordance with an embodiment of the present invention;

FIG. 1C is a rear right perspective view of the multifunctional cleaning and disinfecting device in accordance with an embodiment of the present invention;

FIG. 2 is a front perspective view of the multifunctional cleaning and disinfecting device without the cover in accordance with an embodiment of the present invention;

FIG. 3 is an exploded view of the multifunctional cleaning and disinfecting device without the cover in accordance with an embodiment of the present invention;

FIG. 4A is a front view of the multifunctional cleaning and disinfecting device without the cover in accordance with an embodiment of the present invention;

FIG. 4B is a front right perspective view of the multifunctional cleaning and disinfecting device without the cover in accordance with an embodiment of the present invention;

FIG. 4C is a front left perspective view of the multifunctional cleaning and disinfecting device without the cover in accordance with an embodiment of the present invention;

FIG. 5A is a left-side view of the multifunctional cleaning and disinfecting device without the cover in accordance with an embodiment of the present invention;

FIG. 5B is another front perspective view of the multifunctional cleaning and disinfecting device without the cover in accordance with an embodiment of the present invention;

FIG. 5C is another right perspective view of the multifunctional cleaning and disinfecting device without the cover in accordance with an embodiment of the present invention;

FIG. 6 is a schematic diagram of the multifunctional cleaning and disinfecting device in accordance with an embodiment of the present invention; and

FIG. 7 is a block diagram of the multifunctional cleaning and disinfecting device in accordance with an embodiment of the present invention;

### DETAILED DESCRIPTION OF THE INVENTION

The following description is presented to enable any person skilled in the art to make and use the invention, and is provided in the context of particular applications of the invention and their requirements. Various modifications to the disclosed embodiments will be readily apparent to those skilled in the art and the general principles defined herein may be applied to other embodiments and applications without departing from the scope of the present invention. Thus, the present invention is not intended to be limited to the embodiments shown, but is to be accorded the widest scope consistent with the principles and features disclosed herein. Accordingly, it is intended that the present invention covers such modifications and variations as come within the scope of the appended claims and their equivalents.

In the following detailed description, numerous specific details are set forth in order to provide a thorough understanding of the invention. However, it will be understood by those skilled in the art that the present invention may be practiced without these specific details. In other instances, well-known methods, procedures and components have not been described in detail so as not to obscure the present invention.

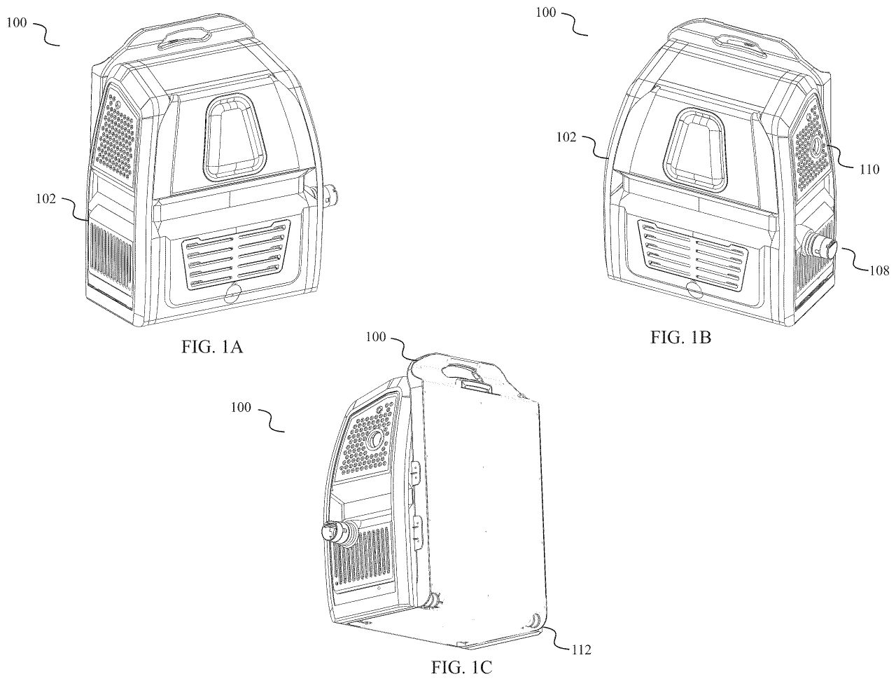

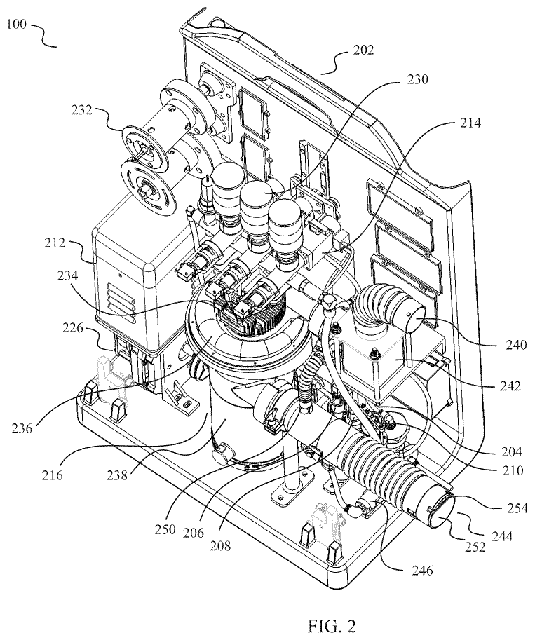

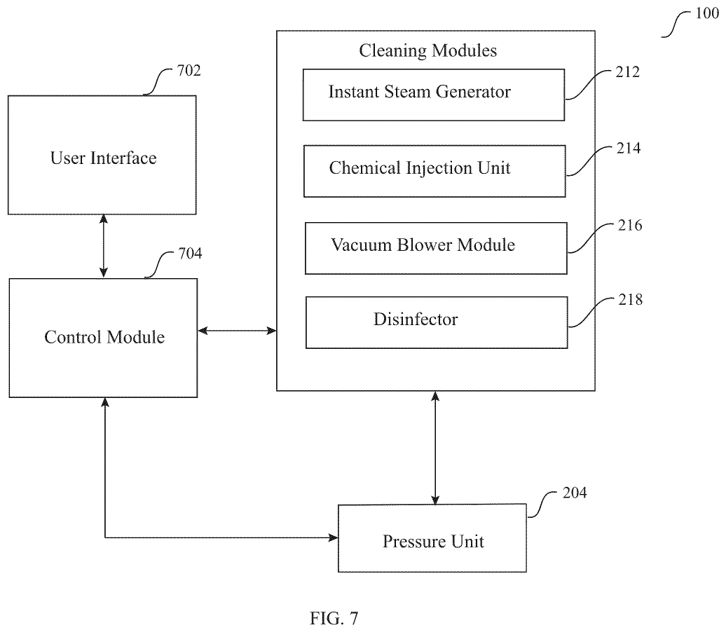

FIG. 1A-1C show the multifunctional cleaning and disinfecting device 100. All the components of the multifunctional cleaning and disinfecting device 100 are contained in the housing 102. The major components of the device 100 are pressure and flow unit 204, a number of cleaning modules and a system control module as illustrated in FIGS. 2, 3 and 6. Reference to FIG. 7, system control module 704 may comprise one or more physical processors programmed with computer program instructions that, when executed by the one or more physical processors, program the system control module 704 to configure the different cleaning modules to perform their respective cleaning and disinfecting functions. The user interface 702 is configured to accept one or more instructions comprising an instruction on a selection of any of the cleaning modules to perform the one or more cleaning and disinfecting functions.

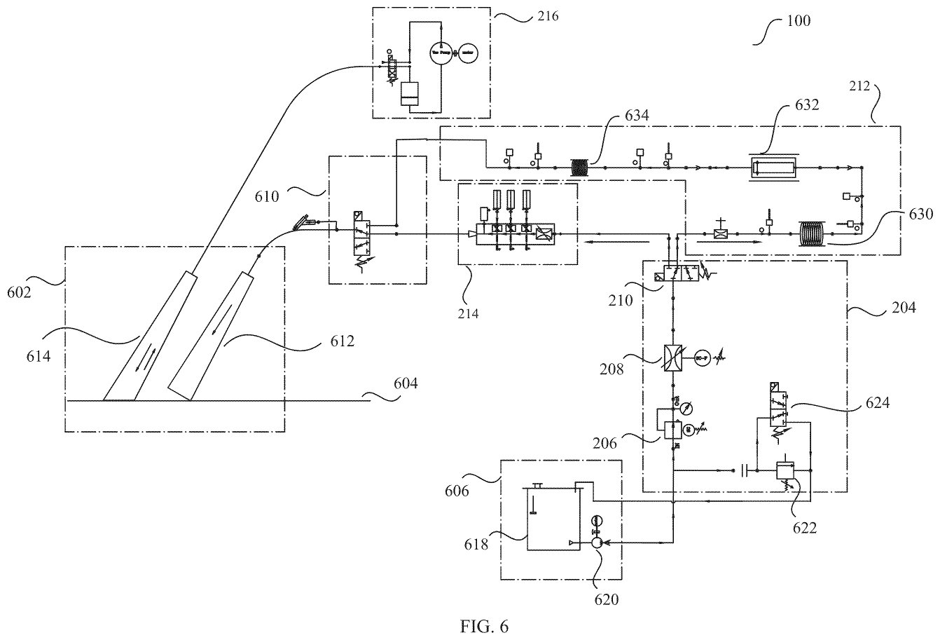

The source of fluid shown in the schematic of FIG. 6 can be included in the housing 102 itself in the form of tank 618 in the tank assembly console 606 or only the pump 620 can be kept inside the housing 102 taking the supply of fluid from an outside source. In either case, a pump motor combination 620 then transfers energy to the incoming fluid. In some embodiments, a reciprocating plunger pump is used which can deliver fluid at a desired high pressure and flow rate. The motor of the motor pump combination 620 of the present invention is a printed-circuit-board (PCB) motor. The pressurized fluid then goes to the pressure and flow unit 204 where the pressure and flow rate is regulated on-demand as set by a user through a user interface. The pressure and flow unit 204 comprises a pressure regulator 206, a flow control valve 208 and a flow diverter 210. An electric motor functionally coupled to the pressure regulator 206 controls it in accordance with the instructions received through the system control module/controller 704. Another smaller and specialized motor moves the stem of the flow control valve 208 to maintain the required flow rate irrespective of the flow pressure based on signal received from a flow meter placed after the flow control valve 208. Accordingly, the pressure and flow unit 204 is configurable to independently set the fluid pressure and flow rate of the fluid corresponding to a selected cleaning function at a desired level from a minimum value to a maximum value. This enables device 100, in one regard, to accomplish several different cleaning functions. For example, the user may independently control the fluid pressure and flow rate of the fluid with the help of the pressure and flow unit 204 to enable the device 100 to selectively discharge the fluid at high pressure when switched to a pressure wash cleaning function or to produce a mist by regulating the flow rate and the fluid pressure when switched to a mister function mode, for example. For each of these example function modes, the device 100 allows a user to independently and dynamically and proportionately set a fluid flow and pressure rate at a defined minimum and maximum value range, where a surface or space is treated by discharge of the regulated fluid passing through the device 100 and selected applicator attachments.

The pressure and flow unit 204 further comprises a system relief valve 622 which protects the system by releasing the fluid in case of accidental excessive pressure build up in the system, and an unloading valve 624 which helps the system run at no load by allowing the fluid to return to the source during idle run of the unit.

Upon selection of the chemical injection cleaning function or the steam cleaning function, the system control module 704 sends instructions to the unloading valve 624 to put the system on a proper load i.e. to pass the fluid through the pressure regulator 206 and flow control valve 208. The fluid leaving the pressure and flow unit 204 has two directions to flow to—either to the chemical cleaning unit 214 or to the steam generator 212. The flow diverter 210 diverts the fluid to either of these two directions as per the user requirement selection. For example, when a user selects a chemical cleaning function from the user interface 702 as shown in FIG. 7, the flow diverter 210 directs the fluid flow toward the chemical cleaning unit 214. If a steam cleaning function is selected next by the user then the system control module 702 actuates the flow diverter 210 to cut off the fluid flow in the direction of chemical cleaning unit 214 and, instead, directs the flow toward the steam generator 212.

The high-pressure instant steam generator 212 comprises a water band heater 630, an evaporator 632 and a super heater 634. The fluid supplied from the high-pressure and flow unit 204 receives heat energy through the water band heater 630 and then gets evaporated to steam in the evaporator 632. The saturated steam is subsequently converted to a superheated steam in the superheater 634. A user can select a steam temperature and flow through the user interface 702 and the controller 704 accordingly sends instructions to adjust the heating to produce the desired steam temperature.

The chemical cleaning unit 214 proportionately mixes various chemicals with the fluid supplied from the pressure and flow unit 204 on-demand in accordance with a selection made from the user interface 702. Chemical compounds or substances in detachable containers are mounted to a manifold of the chemical cleaning unit in fluid communication with the pressure and flow unit 204 through a series of check valve and orifices. The controller 704 controls the check valve to allow flow of right quantity of chemical from the containers as requested on-demand from a user selection Once a desired concentration of chemical is set, the mixing ratio is maintained irrespective of the rate of flow of the fluid coming from the pressure and flow unit 204. The fluid with or without the chemical can be discharged at a fluid pressure and a fluid flow rate set at a desired value between the minimum value and the maximum value for a misting, foaming, spraying and/or fogging cleaning with the help of one or more applicators.

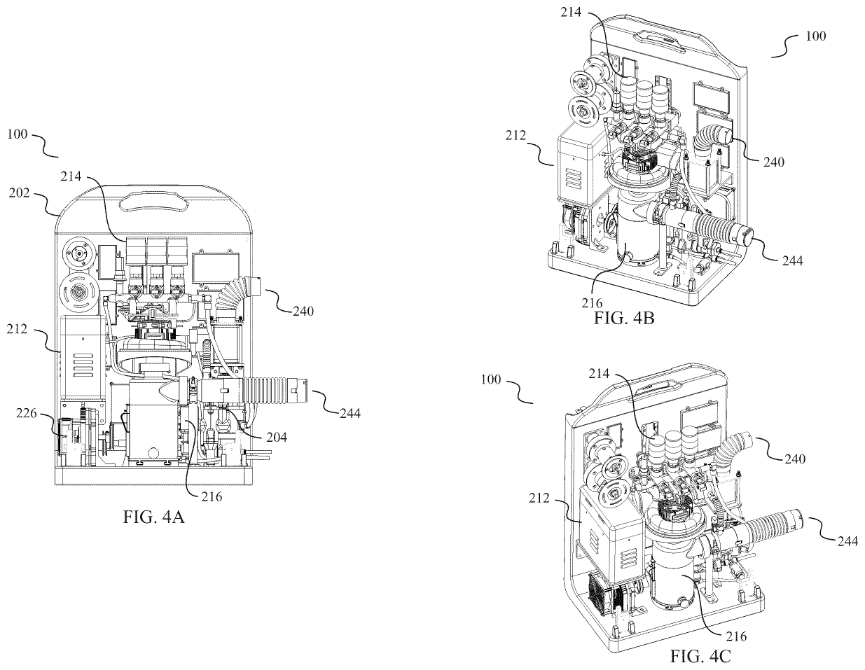

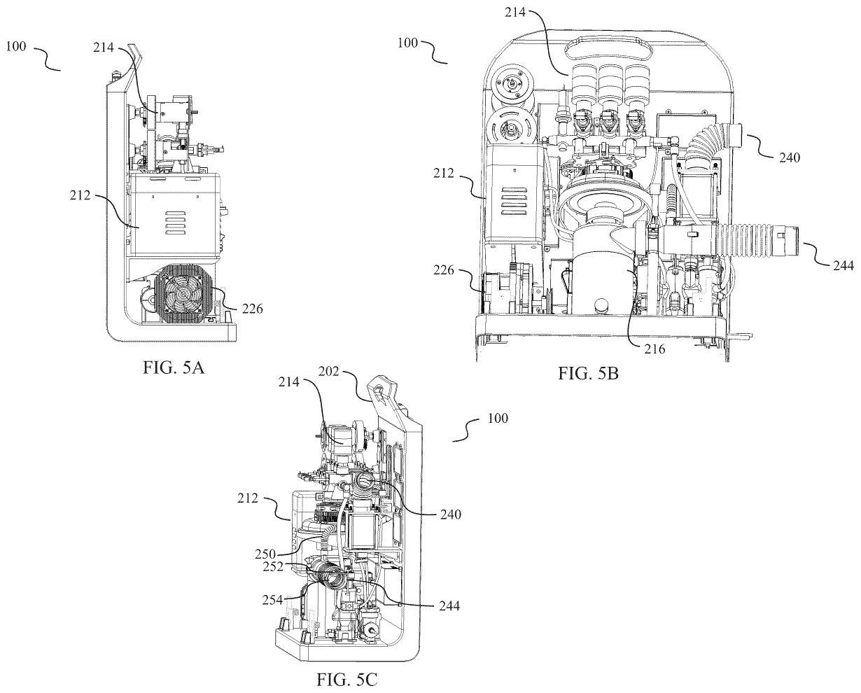

Reference to FIGS. 2-7, the housing 102 also contains a vacuum blower unit 216 configured to selectively generate an adjustable negative pressure or positive pressure. The vacuum blower unit 216 comprises a vacuum blower motor 234, a vacuum blower fan 236 coupled to the vacuum blower motor 234, a pre-separator 238 mounted in fluid communication with the blower fan 236, a blower exhaust 240 positioned downstream to the blower fan 236, a blower vacuum valve 242 disposed in between the blower fan 235 and the blower exhaust 242, a split pipe 244 positioned upstream to the pre-separator 238 and a blower return line 250. The split pipe 244 is longitudinally separated by an internal wall to form two distinct sections-a suction part 252 through which ambient air can enter the vacuum blower unit 216, and a blower part 254 through which pressurized air discharged by the blower fan 236 can exit the vacuum blower unit 216, when needed. Depending on the selection made by a user through the user interface 702, system control module 704 configures the vacuum blower unit 216 to create either a positive air pressure or a negative pressure. If a user wants to use the device 100 as a vacuum unit then the blower vacuum valve 242 gets opened and the negative pressure created in the suction part 252 can be used for vacuum cleaning operation. In the blower mode, the blower vacuum valve 242 gets closed and the blower return line 250 gets opened to direct the air discharged by the blower fan 235 to the blower part 254. This pressurized air can then be used for air blowing cleaning applications. A filter separates the debris from a fluid drawn through a suction inlet and the pre-separator 238 helps in storing the solid particles separately inside the canister such that a relatively clean flow of fluid exits through the blower exhaust 240 via blower valve 242.

Reference to FIG. 6, one or more attachment applicator adapter 602 are provided to extend the flow of fluid/chemical to carry out the above-mentioned cleaning and disinfecting functions on a surface or space 604. The attachment applicator adapter 602 are configured to be removably attachable to the housing 102 so that applicator attachments suitable for a specific purpose can be used when required. For example, one or more hand and floor cleaning and disinfecting attachments may be attached to the attachment adapter. A hose 612 configured to optionally carry and direct the flow of chemical mixed fluid, coming from chemical unit 214 or flow of steam coming from the steam generator 212, as shown in FIG. 6, can be used as an applicator when the chemical cleaning function or steam generation cleaning is chosen. Similarly, applicator 614 can be attached to the attachment adapter 602 when blower or vacuuming functions are selected.

A steam and water cleaning diverter 610 installed after the steam generator 212 and chemical unit 214 directs, by spring pressure, a diverted flow of fluid, from either the steam unit or the chemical unit to flow to the attachment adapter 602, depending on the selection made. The diverter 610, for example, can be a double check valve.

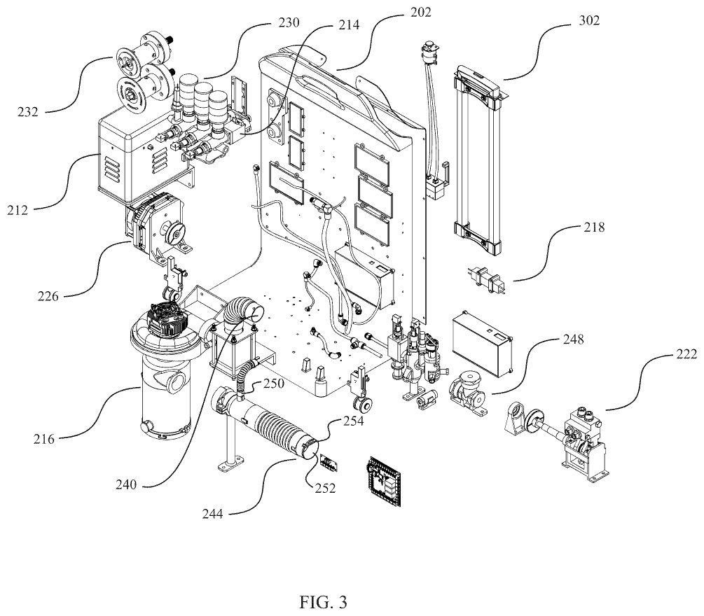

In one embodiment, with reference to FIG. 3, the cleaning modules may include a disinfector 218 configured to deliver an electrically charged aerosol mist from the fluid coming through the pressure and flow unit 204 on the surface or space 604. The disinfector 218 is configured to automatically deliver an effective dose of ultraviolet radiation for antibacterial and antiviral treatment of surfaces. It is configured to enable a user to operate it in different operating modes based on the specific disinfecting requirements. System control module 704 controls functioning of the disinfector 218 based on feedback received from one or more sensors installed at the applicator.

As described above, by making all the cleaning modules compact and having them all housed inside a single housing 102, the multifunctional cleaning and disinfecting device 100 of the present invention serves as a light-weight ultraportable system which eliminates the need to carry many different standalone cleaning equipment to a site. The compact custom design of the cleaning modules, motors, housing, and pressure and flow unit makes the size of the device equivalent to a common luggage suitcase with weight anywhere from 400% to 700% less than that of the weight of the current independent cleaning systems when combined to produce comparable functionality. The system control module 704 coordinates and controls functions of all the components of the device 100 for a specific cleaning need. It also allows interfacing of the built-in user interface 702 of the device 100 with a remote device through an app.

1. A multifunctional cleaning and disinfecting device, comprising:
a housing;
a pressure and flow unit comprising a pressure regulator, a flow control valve and a flow diverter, said pressure and flow unit mounted inside said housing being configured to independently control a pressure and a flow rate of a fluid;
a plurality of cleaning modules housed inside said housing, each of said plurality of cleaning modules being configured to perform one or more cleaning and disinfecting functions on a surface or space;
a user interface configured to accept one or more instructions, said one or more instructions comprising an instruction on a selection of a cleaning module from said plurality of cleaning modules to perform said one or more cleaning and disinfecting functions; and
a system control module configured to control said one or more cleaning and disinfecting functions corresponding to said one or more instructions.

2. The device of claim 1, wherein said plurality of cleaning modules include an instant steam generator configured to generate an adjustable steam flow from said fluid coming through said pressure and flow unit for one of said one or more cleaning and disinfecting functions as per said selection.

3. The device of claim 1, wherein said plurality of cleaning modules include a chemical injection unit configured to dynamically and proportionately mix one or more substances with said fluid coming through said pressure and flow unit for one of said one or more cleaning and disinfecting functions as per said selection.

4. The device of claim 1, wherein said plurality of cleaning modules include a vacuum blower unit configured to selectively generate an adjustable negative pressure or an adjustable positive pressure for one of said one or more cleaning and disinfecting functions.

5. The device of claim 1, wherein said plurality of cleaning modules include a disinfector configured to deliver an electrically charged aerosol mist from said fluid coming through said pressure and flow unit on said surface for one of said one or more cleaning and disinfecting functions.

6. The device of claim 1, further comprising one or more attachment applicator adapters optionally removably attachable to said housing, said one or more attachment applicator adapters being configured to extend release of a flow of said fluid for one of said one or more cleaning and disinfecting functions.

7. The device of claim 1, further comprising a motor pump combination configured to pressurize said fluid through said pressure and flow unit.

8. The device of claim 6, wherein one or more hand and floor cleaning and disinfecting attachments are attachable to said one or more attachment applicator adapters.

9. The device of claim 7, said motor is a printed-circuit-board (PCB) motor.

10. A multifunctional cleaning and disinfecting device comprising:
a housing;
a plurality of cleaning modules housed inside said housing, each of said plurality of cleaning modules being configured to perform one or more cleaning and disinfecting functions on a surface;
a pressure and flow unit housed inside said housing, said pressure and flow unit comprising a pressure regulator, a flow control valve and a flow diverter; and
a system control module housed inside said housing, said system control module comprising one or more physical processors programmed with computer program instructions that, when executed by said one or more physical processors, program said system control module to:
configure, as per a user selection, one or more cleaning modules from said plurality of cleaning modules to perform a cleaning function; and
configure said pressure and flow unit to set a fluid pressure and a fluid flow rate of a fluid corresponding to said cleaning function at a desired value from a minimum value to a maximum value.

11. The device of claim 10, wherein said one or more cleaning modules adjustably mix one or more substances with said fluid coming through said pressure and flow unit if said cleaning function is a chemical injection function.

12. The device of claim 10, wherein one or more cleaning modules generate a steam flow from said fluid coming through said pressure and flow unit if said cleaning function is a steam cleaning function.

13. The device of claim 10, wherein said cleaning function is a pressure wash function with said fluid being discharged at said fluid flow rate set at said maximum value and said fluid pressure set at said minimum value.

14. The device of claim 10, wherein said cleaning function is a misting and fogging function with said fluid being discharged at said fluid pressure and said fluid flow rate set between said minimum value and said maximum value.

15. The device of claim 10, wherein said cleaning function is an electrostatic spraying function with said fluid being discharged at said fluid pressure and said fluid flow rate set between said minimum value and said maximum value.

16. A multifunctional cleaning and disinfecting device, comprising:
a housing;
a plurality of cleaning modules housed inside said housing, each of said plurality of cleaning modules being configured to perform one or more cleaning and disinfecting functions on a surface;
a user interface configured to accept one or more instructions, said one or more instructions comprising an instruction on a selection of a cleaning module from said plurality of cleaning modules to perform said one or more cleaning and disinfecting functions; and
a system control module housed inside said housing, said system control module being configured to control said one or more cleaning and disinfecting functions corresponding to said one or more instructions.

17. The device of claim 16, wherein said plurality of cleaning modules include a vacuum blower unit configured to selectively generate an adjustable negative pressure or an adjustable positive pressure, said vacuum blower unit comprising a vacuum blower motor, a vacuum blower fan coupled to said vacuum blower motor, a pre-separator mounted in fluid communication with said blower fan, a blower exhaust positioned downstream to said blower fan, a blower vacuum valve disposed in between said blower fan and said blower exhaust, a split pipe positioned upstream to said pre-separator and a blower return line.

18. The device of claim 17, further comprising one or more attachment applicator adapter removably attachable to said housing, said one or more attachment applicator adapter being configured to extend a path of an air flow at said adjustable positive pressure or at said adjustable negative pressure.

19. The device of claim 18, wherein one or more hand and floor cleaning and disinfecting attachments are attachable to said one or more attachment applicator adapter.

20. The device of claim 17, wherein said motor is a printed-circuit-board (PCB) motor.
